(19) United States
(12) Patent Application Publication
(10) Pub. No.: US 2025/0375474 A1
(43) Pub. Date: Dec. 11, 2025

(54) COMPOSITIONS FOR THE PREVENTION AND TREATMENT OF MOISTURE-ASSOCIATED SKIN DAMAGE

(71) Applicants: Johanna MERCER, Port Williams (CA); SCOTIADERM INC., Port Williams (CA)

(72) Inventor: Ann GORDON, Port Williams (CA)

(21) Appl. No.: 18/875,919

(22) PCT Filed: Jun. 21, 2023
(86) PCT No.: PCT/CA2023/050866

§ 371 Date: Dec. 17, 2024

Related U.S. Application Data
(60) Provisional application No. 63/354,447, filed on Jun. 22, 2022.

(51) Int. Cl. A61K 33/30; A61K 9/00; A61K 9/06; A61K 31/4174; A61K 31/573; A61K 31/695; A61K 47/02; A61K 47/06; A61K 47/10; A61K 47/18; A61K 47/36; A61K 47/44; A61P 17/16

(52) U.S. Cl. CPC A61K 33/30; A61K 9/0014; A61K 9/06; A61K 31/4174; A61K 31/573; A61K 31/695; A61K 47/02; A61K 47/06; A61K 47/10; A61K 47/183; A61K 47/36; A61K 47/44; A61P 17/16

(57) ABSTRACT

Therapeutic compositions, kits, and methods and uses thereof are provided. The compositions comprise zinc oxide, hydrocortisone, and/or clotrimazole.

### BACKGROUND

Moisture-associated skin damage (MASD) is a term for the spectrum of injuries characterized by inflammation and erosion of the epidermis. Overexposure of skin to moisture can compromise its integrity, and once damaged, the skin is more permeable and susceptible to irritants, such as urine, stool, perspiration, wound exudate, ostomy effluent, and the like. Further, wet skin has a high coefficient of friction, making it susceptible to friction and shear damage. MASD encompasses multiple clinical entities, such as incontinence-associated dermatitis (IAD), intertrigo (ITD), periwound skin damage, and peristomal MASD.

Patients with MASD experience intense, persistent symptoms such as pain, burning, and pruritus, especially where skin breakdown involves partial-thickness erosions and denudement. Emerging evidence highlights the association between MASD and other skin conditions such as dermatitis, cutaneous fungal/bacterial infection, and pressure injuries.

The development and severity of MASD depend on a number of intrinsic and extrinsic factors. It is common among individuals with excessive perspiration, increased dermal metabolism, abnormal skin pH, history of atopy, deep body folds, dermal atrophy, and inadequate sebum production. Extrinsic factors that may precipitate and exacerbate MASD are chemical/biologic irritants, mechanical stress on the skin, fungal/candidiasis proliferation, seasonal or environmental factors, incontinence (urine, fecal, or both), and hygienic practices.

The prevention and treatment of MASD is of great importance for a number of patient populations. The present disclosure provides compositions for this purpose.

### SUMMARY

Compositions and methods are provided for prevention and treatment of moisture-associated skin damage, and including without limitation intertrigo and incontinence associated dermatitis, for human and veterinary use. Compositions, which may be referred to as “Dr. Gordon's Bum Cream” are petrolatum-based, and comprise, consist essentially of, or consist of, as active ingredients, from about 5% to about 20% by weight zinc oxide; from about 0.2% to about 2.5% by weight hydrocortisone; and from about 0.1% to about 1% clotrimazole; and the balance by weight petrolatum. Optionally, dimethicone is present at from about 0.01% to about 2.5% by weight.

In one embodiment, a method is provided for the prevention or treatment of MASD, the method comprising topical administration of an effective dose of a composition comprising, consisting essentially of, or consisting of, as active ingredients, from about 5% to about 20% by weight zinc oxide; from about 0.5% to about 2.5% by weight hydrocortisone; and from about 0.1% to about 1% clotrimazole; and the balance by weight petrolatum. Optionally, dimethicone is present at from about 0.01% to about 2.5% by weight. Volume of the amount administered will vary with the size of surface to be treated, but may generally comprise that amount required for a thin layer of the therapeutic formulation on the skin. Administration may be repeated daily, twice daily, every other day, every third day, every fourth day, weekly, etc., as required. In some embodiments the medicated formulation is administered for a period of time not more than about 10 days, and in some embodiments for not more than about 7 days.

In some embodiments the therapeutic formulation is provided as a lotion. The lotion is optionally packaged for single use, e.g. in a stick pack or the like in a volume of from around 0.1 to 10 ml. Larger, multi-use packaging is also of interest. In some embodiments the formulation is provided embedded on a disposable wipe, which may be packaged for single use, or in a multi-pack format. The therapeutic formulation may also be provided in an aerosol dispenser.

In addition to the healing benefits provided by the formulations of the invention, in which the length of time required for the recovery of the skin is shortened, the formulation of the invention also provides a cosmetically acceptable formulation that does not damage bedding, clothes, etc., thereby allowing continued use over an extended period of time for healing.

### DESCRIPTION OF THE FIGURES

Embodiments of compositions, devices, methods, and kits are described throughout reference to the drawings.

FIG. 1 shows graphs of results from in incontinence-associated dermatitis rabbit model treated with therapeutic cream composed of 1.0% hydrocortisone, 1.0% clotrimazole and 15.0% zinc oxide.

FIG. 2 shows linear regression analysis of enzyme assay test results with the 3% Soybean Extract Cream Samples.

FIG. 3 shows a first test result determining the inhibition by soybean extract with and without cream.

FIG. 4 shows a second test result determining the inhibition by soybean extract with and without cream.

### DETAILED DESCRIPTION OF THE EMBODIMENTS

The compositions of the present invention can help alleviate the impact of MASD and decrease the time for recovery of the skin. The therapeutic formulation is cosmetically acceptable, and provides for improved barrier and healing properties.

Benefits of the formulations described herein include clinically proven, comprehensive healing and protection in a safe, physiologically and cosmetically acceptable formulation. The pH of the formulation is substantially similar to normal skin pH. The formulations are gentle, free of parabens, phthalates, fragrance, alcohol, and are easy to apply. The cosmetically acceptable formulation does not stain fabric. The formulation, once applied, is transparent for easy skin inspection. It stays in place once applied, but is easily removed with gentle cleaning. By providing a cost-effective, single formulation for MASD, the number of products, resources and time required to complete a skin care regimen is minimized.

### Definitions

In the description that follows, a number of terms conventionally used are utilized. In order to provide a clear and consistent understanding of the specification and claims, and the scope to be given to such terms, the following definitions are provided.

Terms and symbols of used herein follow those of standard treatises and texts in the field, e.g. Kornberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); and the like.

The terms “therapeutic agents” or “drugs” can be used interchangeably herein and include the pharmaceutically active compositions of the invention as described herein.

Moisture-associated skin damage (MASD) is a term for the spectrum of injuries characterized by inflammation and erosion of the epidermis. Particular conditions for treatment include, for example, incontinence-associated dermatitis. The ammonia in urine and/or stool creates an alkaline environment that potentiates the proteolytic activity of fecal enzymes (protease and lipase) on skin, leading to IAD. These enzymes disrupt the skin acid mantle, making it easy for irritants to penetrate into the skin and trigger an inflammatory response. Current estimates of IAD prevalence ranges from 5.6% to 50% across different healthcare settings, patient populations, and age groups; it is highest among critically ill patients.

Intertriginous Dermatitis, also called intertrigo, ITD is a type of moisture-related skin damage between skin folds, commonly found in the inframammary, pannus, groin, perianal, and interdigital areas. The combination of excess moisture from perspiration, occlusion with limited air flow, and friction between the opposing epidermal surfaces can lead to ITD. Intertriginous dermatitis is initially characterized by mirror-image erythema, inflammation, and peripheral scaling, but over time the epidermis can become macerated, edematous, crusted, and eroded and provide an optimal environment for the proliferation of microorganisms such as Candida albicans that can cause secondary infections.

Periwound Skin Damage. Wound fluid contains endogenous protein-degrading enzymes that are caustic and damaging to the intact skin. Periwound skin is particularly vulnerable to MASD when drainage volume exceeds the fluid-handling capacity of a dressing. In addition, repetitive application and removal of adhesive tapes and dressings may strip away the periwound stratum corneum, precipitating further skin damage.

Peristomal Moisture-Associated Skin Damage is characterized by inflammation and erosion of the mucocutaneous junction and surrounding area. Despite various containment strategies, fecal effluent may leak and spill over to the peristomal skin, particularly in patients with hyperactive bowels, diarrhea, and fistulas that connect the bowel and skin surface. Undulating contour of the abdomen from excessive subcutaneous fat, poor muscle tone, herniation, fissures (a linear break in the skin with a dermal base), or crevices linked to skin/muscle defects present challenges that often lead to poor appliance adherence and pouch leakage. Establishing a secure pouching system postmaceration is the primary complication associated with peristomal MASD, because it perpetuates a vicious cycle: Eroded epidermis produces moisture that impedes the pouching system from adhering to the skin and forming a tight seal, leading to further effluent-skin contact that in turn causes greater maceration and pouching difficulties.

The severity of MASD, and the improvement thereof maybe assessed by various clinical indicia. Administration of the therapeutic formulations of the present disclosure reduce one or more of these indicia, e.g. by lowering a score in a standardized test by at least about 10%, at least about 25%, at least about 50%, or more.

For examples, Incontinence-associated dermatitis typically presents as diffuse erythema but may also be characterized by erosion, edema, scaling, papules, or bullae containing serous exudate with accompanying pruritus, burning, or pain. The Incontinence Associated Dermatitis and Its Severity instrument is a tool that assesses for redness, skin loss, and rash in the 13 body locations affected by IAD. A score of 0 to 52 is generated and used to inform practice. Further, the Incontinence-Associated Dermatitis Skin Condition Assessment Tool was developed by Beeckman et al describe the surface area (in centimeters squared), severity of redness, and depth of any perineal skin lesion.

More recently, a Global IAD Categorization tool was developed. First, the damaged skin is assessed to determine whether persistent redness or skin loss is present. Next, clinical infection or intertrigo is evaluated based on a cluster of signs and symptoms. As such, the IAD is classified into 4 categories: persistent redness without clinical signs of infection, persistent redness with clinical signs of infection, skin loss without clinical signs of infection, and persistent redness with clinical signs of infection.

While ITD and IAD are precipitated by similar factors, ITD affects other areas that are not affected by incontinence. Areas affected by ITD can appear erythematous with scaling. Secondary candida intertrigo is plausible based on the characteristic appearance of satellite lesions. However, a validated measurement scale to describe the severity of ITD is not available.

Periwound skin damage is evident from the varying degree of skin maceration, erythema, edema, inflammation, blistering, excoriation, and erosion. White maceration is when the skin appears white and swollen, and erythematous maceration is when the skin is reddened and inflamed. Characteristic manifestations of periwound skin damage include erosion, erythema, edema, bleb formation, pruritus, edema, and pain.

Peristomal MASD is inflammation and erosion of the skin related to moisture that begins at the stoma/skin junction and may extend outward. The Ostomy Skin Tool is designed to assess the peristomal skin in 2 ways. First, it determines a score based on discoloration, erosion, and tissue overgrowth. Pictorial references are provided to aid the assessor. Second, the Ostomy Skin Tool provides a diagnostic guide that directs the caregiver through an interview with the patient to determine possible causes of the skin disorder (eg, chemical irritation, mechanical irritation, or infection).

Urinary incontinence: The principal effects of urinary incontinence on affected skin relate to the chronic overexposure to moisture and the usually elevated pH of urine. Skin pH is typically in the range of 5.0 to 5.9 for healthy skin. This pH supports healthy skin physiology and is essential for normal skin processes. It has been shown that exposure to elevated pH (from urine or cleansers formulated at higher pH), can lead to skin damage due to factors such as elevated water loss from disrupted barrier function and interruption of stratum corneum production.

Fecal incontinence: Along with bile acids, waste products and bacteria, digestive enzymes are typically found in fecal matter, as they are progressing through the intestinal tract as expected. These digestive enzymes can come from many different classes of enzyme (i.e. lipases which digests fats, proteases which digests proteins, etc.), each with a specific desired biological role. They act by cleaving/breaking key bonds in food, into smaller units, as part of the digestive process. However, when these enzymes are present in unintended areas or tissues such as on the skin, or in wounds, they can cause significant damage by cleaving unintended targets in other tissues that would not normally be exposed to these enzymes. This enzyme activity can cause new damage and prevent effective healing in wounded skin and can provide an environment that fosters microbial growth, as some of the smaller units formed as part of the digestion can act as food for microbes.

Some examples of digestive proteases that can be found in fecal matter, are chymotrypsin, trypsin and pancreatic elastase, all of which help digest proteins by cleaving after different positions in protein chains. As well, other enzymes, such as lipases, which normally assist in the digestion of fats, can also be present. Although these are normally expected to be present in varying degrees, in fecal matter, when fecal matter is in contact with wounds or for long periods of time with skin, these enzymes can cleave unintended targets, leading to new or worsened skin damage.

Combined urinary and fecal incontinence: When both urine and fecal matter are present simultaneously, beyond their individual effects on skin and skin structure, the elevated pH of the urine can further accentuate the enzymatic activity of the digestive enzymes, as many of these have maximal activity in the basic pH (>7). For example, the pH maximums for trypsin (7.8 to 8.7) and pancreatic lipase (8.0) are in the range of normal to alkaline urine pH levels (4.5 to 8.0 and above).

Biofilm formation is an established characteristic of microbial infections. As a microbial (fungal, bacterial, etc.) infection matures, formation of a protective matrix, that helps support the microbial colonies, a biofilm, can result. This biofilm matrix can not only hinder wound healing but can also prevent or interfere with antimicrobial compounds and/or the body's own immune response factors, from reaching their targets, to help clear out the infection. Ideally, biofilm production should be minimized in a wound, to lead to overall better outcomes.

The terms “subject,” “individual,” and “patient” are used interchangeably herein to refer to a mammal being assessed for treatment and/or being treated. In an embodiment, the mammal is a human. The terms “subject,” “individual,” and “patient” encompass, without limitation, individuals having or at risk of having an adhesion. Subjects may be human, but also include other mammals, particularly those mammals useful as laboratory models for human disease, e.g. mouse, rat, etc.

The term “diagnosis” is used herein to refer to the identification of a pathological state, disease or condition, such as the identification of MASD and subtypes therein.

As used herein, the terms “treatment,” “treating,” and the like, refer to administering an agent, or carrying out a procedure, for the purposes of obtaining an effect in a subject (e.g., a patient). The effect may be prophylactic in terms of completely or partially preventing an undesirable condition, disease or symptom thereof and/or may be therapeutic in terms of effecting a partial or complete remedy, remission, or cure for an undesirable condition, disease and/or symptoms of the disease. Treating thus encompasses the administration of a formulation before an undesirable condition, disease or symptom thereof occurs, during the development of an undesirable condition, disease or symptom thereof, and/or after an undesirable condition, disease or symptom thereof has occurred. Treating may refer to any indicia of success in the treatment or amelioration or prevention of an undesirable condition or disease, including any objective or subjective parameter such as abatement; remission; diminishing of symptoms or making the undesirable condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of the formulation of the present disclosure to prevent or delay, to alleviate, or to arrest or inhibit development of the symptoms or undesirable condition. The term “therapeutic effect” refers to the reduction, elimination, or prevention of the undesirable condition, disease, or symptoms or side effects of thereof.

“In combination with”, “combination therapy” and “combination products” refer, in certain embodiments, to the concurrent administration to a patient of a first therapeutic and a second therapeutic. When administered in combination, each component can be administered at the same time or sequentially in any order at different points in time. Thus, each component can be administered separately but sufficiently closely in time so as to provide the desired therapeutic effect.

As used herein, the term “correlates,” or “correlates with,” and like terms, refers to a statistical association between instances of two events, where events include numbers, data sets, and the like. For example, when the events involve numbers, a positive correlation (also referred to herein as a “direct correlation”) means that as one increases, the other increases as well. A negative correlation (also referred to herein as an “inverse correlation”) means that as one increases, the other decreases.

“Dosage unit” refers to physically discrete units suited as unitary dosages for the particular individual to be treated. Each unit can contain a predetermined quantity of active compound(s) calculated to produce the desired therapeutic effect(s) in association with the required pharmaceutical carrier. The specification for the dosage unit forms can be dictated by (a) the unique characteristics of the active compound(s) and the particular therapeutic effect(s) to be achieved, and (b) the limitations inherent in the art of compounding such active compound(s).

“Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

The terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects to a degree that would prohibit administration of the composition.

A “therapeutically effective amount” means the amount that, when administered to a subject, is sufficient to effect treatment of an undesirable condition, symptom, or disease of the subject. The effective dose is sufficient to reduce adhesions by at least about 10%, at least about 20%, at least about 30%, at least about 40%, at least about 50%, at least about 60%, at least about 70%, at least about 80%, at least about 90%, or more. The dose of a therapeutic antibody, for example, may be from about 1 mg/kg body weight to about 100 mg/kg body weight, and may be administered immediately following surgery or other event expected to result in adhesion formation; or for reducing existing adhesions. Dosing may be repeated daily, every 2 days, every 3 days, semi-weekly, weekly, etc.

### Detailed Description of the Embodiments

As summarized above, the present disclosure provides methods for treating adhesions in a subject, which includes, e.g., preventing adhesion formation, halting or reducing the formation of adhesions, and/or reversing or eliminating established adhesions in a subject. Compositions and kits are also provided for performing such methods.

Before aspects of the present invention are described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Formulation

Therapeutic formulations, which may be referred to as “Dr. Gordon's Bum Cream” (DGBC) are petrolatum-based, and in some embodiments comprise, consist essentially of, or consist of, as active ingredients, from about 5% to about 20% by weight zinc oxide; from about 0.2% to about 2.5% by weight hydrocortisone; and from about 0.1% to about 1% clotrimazole; and the balance by weight petrolatum. Optionally, dimethicone is present at from about 0.01% to about 2.5% by weight. In formulating, the zinc oxide may be admixed with petrolatum, optionally with dimethicone, to make a paste. The hydrocortisone and clotrimazole powders are then mixed into the paste for the final formulation.

In other embodiments, the therapeutic formulations comprise, consist essentially of, or consist of, as active ingredients, from about 5% to about 20% by weight zinc oxide; from about 0.2% to about 2.5% by weight hydrocortisone; from about 0.1% to about 1% clotrimazole, and from; about 30% to 60%, preferably about 40% to 50%, by weight petrolatum; and the remainder as non-active ingredients.

Zinc oxide (CAS Number 1314-13-2) is an inorganic compound with the formula ZnO. It is a white powder that is insoluble in water. Although it occurs naturally as the mineral zincite, most zinc oxide is produced synthetically. By weight, zinc oxide is present at a concentration of from about 5%, about 6%, about 7%, about 8%, about 9%, about 10% and up to about 20%, about 19%, about 18%, about 17%, about 16%, about 15%. In some embodiments zinc oxide is present at a concentration by weight of from about 10% to about 15%, from about 11% to about 14%, from about 12% to about 13%, and may be present at a concentration by weight of about 12.5%.

Hydrocortisone, 11β, 17α,21-Trihydroxypregn-4-ene-3,20-dione (CAS Number: 50-23-7) is a synthetic or semisynthetic analog of natural hydrocortisone hormone produced by the adrenal glands with primary glucocorticoid and minor mineralocorticoid effects. As a glucocorticoid receptor agonist, hydrocortisone promotes protein catabolismgluconeogenesis, capillary wall stability, renal excretion of calcium, and suppresses immune and inflammatory responses. By weight, hydrocortisone is present at a concentration of from about 0.2%, about 0.3%, about 0.4%, about 0.5%, and up to about 2.5%, about 2%, about 1.5%, about 1%. In some embodiments, hydrocortisone is present at a concentration by weight of from about 0.2% to about 0.5%. In other embodiments hydrocortisone is present at a concentration by weight of from about 0.5% to about 1.5%; from about 0.75% to about 1.25%, and may be present at a concentration by weight of about 1%.

Clotrimazole (CAS Number 23593-75-1) is a synthetic, imidazole derivate with broad-spectrum, antifungal activity. Clotrimazole inhibits biosynthesis of sterols, particularly ergosterol, an essential component of the fungal cell membrane, thereby damaging and affecting the permeability of the cell membrane. By weight, clotrimazole is present at a concentration of from about 0.1%, about 0.2%, about 0.3%, about 0.4%, and up to about 1%, about 0.9%, about 0.8%, about 0.7%. In some embodiments, clotrimazole is present at a concentration by weight of from about 0.25% to about 0.75%; from about 0.4% to about 0.6%, and may be present at a concentration by weight of about 0.5%.

Dimethicone (polydimethylsiloxane; CAS number 63148-62-9) acts as an anti-foaming agent, emollient, and skin protectant. It prevents water loss by forming a hydrating barrier on the skin. Dimethicone may be absent or present. If present, by weight, dimethicone is present at a concentration of from about 0.01%, about 0.02%, about 0.03%, about 0.04%, and up to about 2.5%, about 2%, about 1%, about 0.5%. In some embodiments, dimethicone is present at a concentration by weight of from about 0.025% to about 0.075%; from about 0.04% to about 0.06%, and may be present at a concentration by weight of about 0.05%.

In one embodiment, the therapeutic formulations comprise 1.0% hydrocortisone, 1.0% clotrimazole and 15.0% zinc oxide.

Petrolatum (CAS Number 8009-03-8) is a complex combination of hydrocarbons obtained as a semi-solid from dewaxing paraffinic residual oil. It consists predominantly of saturated crystalline and liquid hydrocarbons having carbon numbers predominantly greater than C25. Petrolatum makes up the balance of the formulation, and so is typically present at from about 80% to 90% by weight, depending on the specific range of the other components.

In some embodiments, non-active ingredients include, but are not limited to, cetearyl alcohol, mineral oil, lanolin, paraffin, water, sodium phosphate, EDTA, modified corn starch, chlorocresol.

In one embodiment, the pH of the formulation is adjusted to about 6.5-7.5 using sodium hydroxide.

In some embodiments, the formulation comprise additives, such as but not limited to: enzyme inhibitors, pH buffers, moisturizing agents or humectants, antimicrobial agents, or antifungal agents. Examples of enzyme inhibitors include, but are not limited to, inhibitors of chymotrypsin, trypsin, pancreatic elastase, or lipase. In one embodiment, enzyme inhibitors comprise fecal enzyme inhibitors. Example fecal enzyme inhibitors include botanicals, such as Balmex Adult Advantage Cream (Active ingredients 11.3% Zn Oxide, Inactive Ingredients: Beeswax, benzoic acid, dimethicone, fragrance, glycine soja (soybean) oil, magnesium aspartate, methylparaben, microcrystalline wax, mineral oil, Oenothera biennis (evening primrose) seed extract, olea europea (olive) leaf extract, panthenol, potassium aspartate, potassium hydroxide, propylparaben, sarcosine, sodium cocoyl amino acids, sorbitan sesquioleate, synthetic beeswax, tocopherol), soybean Bowman-Birk Inhibitor naturally sourced protease inhibitors. Examples of pH buffers include acidic buffers, such as citrates. In some embodiments comprising pH buffers, the buffer maintains the pH between 5.0-5.9 given that alkaline pH promotes skin breakdown and fecal protease activity. Examples of moisturizing agents or humectants include, but are not limited to, hyaluronic acid or polyglutamic acid. Examples of antimicrobial agents include silver bound PGA nanoparticles. Many essential oils are antifungal or antimicrobial and don't trigger the same side effects as some synthetic ingredients. Examples of antifungal and/or antibacterial agents include essential oils from thyme, cinnamon, oregano, clove, mint, citronella, geranium, lemongrass, eucalyptus, peppermint, or grosso lavender; as well as, tea tree oil, clove oil, clary sage oil, jasmine oil, lavender oil, Ylang-ylang oil, eucalyptus oil, cinnamon oil, lemon oil, lemongrass oil, thieves oil, wild oregano oil, jojoba oil, or Manuka tree oil. In one embodiment, the formulation comprise both oil of oregano and lavender. Plant based extracts have antiseptic, anti-inflammatory, astringent, and fungicidal properties. Some can stimulate new cell growth. These properties work together to form a natural treatment for fungal infections on skin. Some moisture associated skin damage conditions include: ringworm, athlete's foot, tinea versicolor, toenail fungus, and jock itch.

In some embodiments, the formulation comprise a proteolytic enzyme inhibitor. Families of proteolytic enzyme inhibitors have been isolated from a wide variety of plants and legumes. Broadly referred to as plant-based inhibitor peptides, these plant-based inhibitor peptides reduce the proteolytic activities of trypsin and chymotrypsin. Exemplary plant-based inhibitor peptides include, but are not limited to: Soybean Tyrosine Inhibitor (STI), Birk-Bowman Inhibitor (BBI), Potato inhibitor I, Potato in hibitor II, Pumpkin trypsin inhibitors, alpha-amylase and trypsin inhibitors from cereals, mustard trypsin inhibitors, serpins, phytocystatins, carboxypeptidase inhibiors, cyclotides, alpha-hairpin inhibitors. In some embodiments, the proteolytic enzyme inhibitor is a tyrosine inhibitor (See Table A). In some embodiments, the proteolytic enzyme inhibitor comprises Soybean Tyrosine Inhibitor (STI), Birk-Bowman Inhibitor (BBI), non-denatured soybean extract, or a combination thereof. In some embodiments, the formulation or compositions described herein comprise 0.1-30%, 1-25%, 0.5-3%, 5-15%, 7-15%, about 3%, about 7%, or about 15% by weight the proteolytic enzyme inhibitor. In some embodiments, uses of a proteolytic enzyme inhibitor, such as a tyrosine inhibitor, is provided for prevention or treatment of MASD. In some embodiments, a composition comprising a a proteolytic enzyme inhibitor (such as a tyrosine inhibitor) and a is provided carrier, for use in the prevention or treatment of MASD. The composition may further comprise clotrimazole, hydrocortisone, and/or zinc oxicde. The composition may be a sprayable composition (wet or dry spray formulations), optionally comprising zinc oxide.

TABLE A
families of protease inhibitors identified in plants

| | Protease | |
|---|---|---|
| | inhibitor | Botanical |
| Num | families | family |
| I | Soybean trypsin | Leguminosae, |
| | inhibitor (SKTI) | Gramineae, Salicaceae, |
| | | Sterculiaceae, Cruciferae, |
| | | Alimataceae, Araceae, |
| | | Solanaceae, Chenopodiaceae, |
| | | Caricaceae, Convolvulaceae |
| II | Bowman-Birk | Leguminosae, Gramineae, |
| | inhibitor (BBI) | Compositae, Alliaceae |
| | Potato inhibitor I | Solanaceae, Gramineae, |
| | | Leguminosae, |
| | | Cucurbitaceae, |
| | | Amarantheaceae, Poligonaceae, |
| | | Lineaceae, |
| | | Compositae, Convolvulaceae |
| IV | Potato inhibitor II | Solanaceae |
| V | Pumpkin trypsin inhibitors | Cucurbitaceae |
| VI | α-amylase and trypsin | Gramineae |
| | inhibitors from cereals | |
| VII | Mustard trypsin inhibitors | Cruciferae |
| VIII | Serpins | Graminae, Cucurbitaceae, Cruciferae |
| IX | Phytocystatins | Poaceae, Curcubitaceae |
| X | Carboxypeptidase | Solanaceae |
| | inhibitors | |
| XI | Cyclotides (cysteine- | Rubiaceae, Violaceae, |
| | rich cyclic peptides) | Fabaceae, Solanaceae, Poaceae |
| XII | α-hairpin inhibitors | Plantaginaceae |

In some embodiments, the formulation comprise Soybean Tyrosine Inhibitor (STI) at concentration of 0-25% by weight (i.e. 1% by weight); Birk-Bowman Inhibitor (BBI), Birk-Bowman Related Protease Inhibitors, and/or Birk-Bowman Family Protease Inhibitors at concentration of 0-25% (i.e. 1% by weight); or non-denatured soybean extract (i.e. 10% by weight). Liquid stool and diarrhea are associated with an increased risk for MASD when fecal materials remain in contact with the skin for a prolonged period. Diarrhea is associated with an increased likelihood of incontinence-associated dermatitis in children and clinical experience strongly suggests that exposure to liquid stool is often associated with severe MASD and extensive erosion of affected skin. Liquid stool contains higher concentrations of proteolytic enzymes such as trypsin and chymotrypsin with the potential to impair the humectant effects of filaggrin and the emollient effects of the intrinsic lipids in the stratum corneum. The deleterious effects on the skin are exacerbated by a more alkaline pH and the higher concentrations of active fecal enzymes associated with diarrhea. Soybeans without precipitation and fermentation produced serious gastric distress, due to the inhibitory activity of Soybean Trypsin Inhibitor (STI) and Birk-Bowman inhibitors (BBI), which block the action of trypsin, chymotrypsin and other proteases needed for protein digestion. It is the heat inactivation of STI and BBI during soybean processing that renders the soybean edible. BBI reduces the proteolytic activities of trypsin, chymotrypsin, elastase, cathepsin G, and chymase, serine protease-dependent matrix metalloproteinases, urokinase protein activator, mitogen activated protein kinase, and PI3 kinase, and upregulates connexin 43 (Cx43) expression.

### Product Use, Form, and Packaging

In use, a quantity of the composition, for example from 1 to 100 ml, is applied to a site of interest from a suitable container or applicator and, if necessary, it is then spread over and/or rubbed into the site using the hand or fingers or a suitable device. Sites for resurfacing techniques include various regions of skin. Reapplication is desirable as required, and treatment may continue for one day, three days, 5 days, 7 days, 10 days.

The therapeutic composition of the invention can be formulated in any form suitable for application to the site of interest, typically as a lotion. The composition can be packaged in any suitable container to suit its viscosity and intended use by the consumer. For example packaged in a bottle, or a propellant-driven aerosol device or a container fitted with a pump suitable for finger operation. When the composition is a gel, it can simply be stored in a non-deformable bottle or squeeze container, such as a tube or a lidded jar. The invention accordingly also provides a closed container containing a cosmetically acceptable composition as herein defined. The closed container may comprise, for example, a tube containing 15, 30, 50, or 100 grams of the therapeutic formulation.

The following examples are put forth so as to provide those of ordinary skill in the art with a complete disclosure and description of how to make and use the subject invention, and are not intended to limit the scope of what is regarded as the invention. Efforts have been made to insure accuracy with respect to the numbers used (e.g. amounts, temperature, concentrations, etc.) but some experimental errors and deviations should be allowed for. Unless otherwise indicated, parts are parts by weight, molecular weight is weight average molecular weight, temperature is in degrees centigrade, and pressure is at or near atmospheric.

### Example A—Formulation 1

| Compound | Percent by weight |
|---|---|
| Zinc oxide (CAS Number 1314-13-2) | 12.5%. |
| Hydrocortisone (CAS Number: 50-23-7) I | 1%. |
| Clotrimazole (CAS Number 23593-75-1) | 0.5%. |
| Dimethicone (CAS number 63148-62-9) | 0.05%. |
| Petrolatum (CAS Number 8009-03-8) | 85.95% |

### Example B—Formulation 2

| Compound | Percent by weight |
|---|---|
| Petrolatum | 45% |
| Cetearyl alcohol and ceteareth-20 | 4% |
| Mineral oil USP | 11% |
| Lanolin USP | 2% |
| Parafin | 4.5% |
| Purified water | 10% |
| Sodium phosphate, dibasic heptahydrate | 0.2% |
| USP | |
| Disodium EDTA | 0.2% |
| Modified corn starch | 6% |
| Zinc Oxide | 15% |
| Clotrimazole | 1% |
| Hydrocortisone | 1% |
| Chlorocresol | 0.1% |

The formulation utilizes a safe but effective low dose of active agents that address common issues of moisture damaged skin. Proper management of skin damaged by IAD (Incontinence Associated Dermatitis) and Intertrigo decreases the risk for the development of pressure injury, and the cream addresses both MASD conditions. Conventional protocols require the sequential use of individual agents, while the single formulation of the present invention requires only a single application, reducing the need for expert care. In short, the formulation provides a simple, safe, effective single-step skin care protocol.

The non-active agents, i.e. zinc oxide and emolliants, keep the actives in contact with skin and provide a barrier. They effectively hold the active agents ‘in place’ so that they can provide healing rather than rubbing off on sheets and clothing, resulting in wasted time and product and slowed or ineffective healing. Nurses and physicians have noted the healing time can be two times faster than the conventional use of single agents. The emollient and zinc also protect the skin from damaging moisture that can recur after treatment, and which can flush active agents from the skin, which would then require reapplication. The emollient and zinc oxide moisturize the skin while protecting it. This provides soothing relief to the patient. Further, less product is required than if multiple products are used. It can be packaged in small amounts for travel or economy. There is a combined saving of both product and the nursing/caregiver time required to treat a patient with an effective, single step product. A simple, effective, single-step solution is important to address complex, chronic and recurring skin care issues.

The compounded mixture is blended to be very gentle and light, it's very easy to put on and remove. This results in less patient discomfort and skin damage due to handling, rubbing and pulling. It is blended to be transparent or ‘vanishing’ for easy inspection.

In an alternative formulation, the cream is layered or embedded on a disposable wipe. Wipes deliver the same one-step process in a single package. One-step incontinence care helps to reduce caregiver time and effort by combining three steps in one. It's efficient. Delivery doesn't require direct contact with the skin by a caregiver, or latex. It is an ideal, hygienic solution for application to avoid direct contact with the risk of transmission of infection or other contamination, or contact with latex in the case of allergy.

Wipes are provided for institutional, home use and also easy to pack in purse, pocket while on day trips. A wipe conveniently provides prescribed dosage, even, controlled distribution, with no rubbing, not sticky, moisture saturated ease of application. For a wipe formulation, dimethicone and aloe vera may be present. In some formulations a biodegradable wipe is provided.

In other embodiments an Aerosol or Spray Pump or Continuous Spray Bottle formulation is provided. Aerosol spray is an effective, sanitary delivery for very tender, damaged skin, and can be provided as a dry or wet spray, foam or gel. Very applicable for medical products. This formulation can be hermetically sealed, to help prevent infection and contamination for both patient and caregiver. As it requires no contact, this helps prevent patient pain and further skin breakdown, and provides even distribution of medication across surface area. Aerosols can be applied in any direction, on any part of the body. It is easy to deliver the right amount exactly where it's needed in a resource efficient manner. An aerosol can also be applied to a cleansing 3-in-1 wipe to add medication when required. Aerosol or spray controls the particle size, spray pattern and concentration for maximum effectiveness and so is cost-effective. Aerosol containers are provided in institutional and travel sizes.

In some embodiments, the therapeutic formulation is provided a sprayable composition comprising one or more spray components. As used herein, “spray components” refer to spraying agents that are an added component or components, with a specific purpose of aiding in spraying as a liquid, gel or foam. The spraying component may evaporate after spraying, gelling or foaming is complete. The spraying component may be inactive or may have complementary activity, such as anti-infective and anti-inflammatory properties, that may be additive or synergistic with the active ingredients of the present invention. Certain spraying components, such as lower alcohols, may act as penetration enhancers and increase transdermal delivery of active components. Exemplary spray components include, but are not limited to, gelling agents, foaming agents, or spray-supporting solvents. In some embodiments, the formulation is sprayable as a liquid, gel, foam, or powder form.

A gelling agent is included in the composition to improve gel formation and stability. In one or more of the embodiments, gelling agents include natural gums tragacanth, carrageenan, pectin, agar and alginic acid; semisynthetic materials such as methylcellulose, hydroxyethyl cellulose, hydroxypropylmethyl cellulose and carboxymethylcellulose and polymers such as carbopols (ex. CARBOPOL® 940). These may be water-based or hydroalcoholic. In one embodiment, one or more gelling agents are independently selected from the group consisting of natural gums tragacanth, carrageenan, pectin, agar and alginic acid; semisynthetic materials such as methylcellulose, hydroxyethyl cellulose, hydroxypropylmethyl cellulose and carboxymethylcellulose and Carbopol polymers. Exemplary gelling agents are also described in U.S. Pat. No. 4,534,958 and EP 0679309, the entire contents of which are incorporated herein by reference.

A foaming agent is included in the composition to improve foaming capability of the formulation and to increase stability of the foam for the intended purpose(s). In one or more embodiments, foaming agents include fatty alcohols, fatty acids, and mixtures thereof. The foaming agent can include at least one fatty alcohol and at least one fatty acid. Suitable fatty alcohols include alcohols having 15 or more carbons in their carbon chain, such as cetyl alcohol and stearyl alcohol (or mixtures thereof). Other examples of fatty alcohols are arachidyl alcohol (C20), behenyl alcohol (C22), 1-triacontanol (C30), as well as alcohols with longer carbon chains (up to C50). Fatty alcohols that are derived from beeswax, including a mixture of alcohols, a majority of which has at least 20 carbon atoms in their carbon chain, are especially well suited as foam adjuvants according to the present invention. In one embodiment, one or more foaming agents are independently selected from the group consisting of fatty alcohols, fatty acids, and mixtures thereof. Exemplary foaming agents are also described in EP 0613728, U.S. Pat. Nos. 4,534,958, 6,126,920 and 8,336,737, the entire contents of which are incorporated herein by reference.

Suitable fatty acids include acids having 16 or more carbons in its carbon chain, such as hexadecanoic acid (C16) stearic acid (C18), arachidic acid (C20), behenic acid (C22), octacosanoic acid (C28), as well as fatty acids with longer carbon chains (up to C50), or mixtures thereof. The concentration of the fatty alcohols and fatty acids required to support the foam system is generally, inversely related to their carbon chain length. Optionally, the carbon atom chain of the fatty alcohol or the fatty acid may have at least on site of unsaturation, may be branched or optionally substituted with a hydroxyl group such as in 12-hydroxy stearic acid. In some embodiments, the foaming agent may include a mixture of fatty alcohols, fatty acids and hydroxy fatty acids and alcohols in any proportion. The total amount of foam adjuvants is about 0.1% to about 5% (w/w) of the carrier mass, and typically, the total amount is about 0.4% to about 2.5% (w/w) of the carrier mass.

A spray-supporting solvent is included in the composition to improve flow characteristics, droplet size, misting capabilities to allow for liquid spraying. The spray-supporting solvent may be volatile to support evaporation upon spraying or after application to the treatment area. In one or more embodiments, spray-supporting solvents include water, a lower alcohol with up to 5 carbons, such as ethanol and isopropyl alcohol, a lower ketone with up to 5 carbons, such as acetone, methylethylketone and 3-pentanone, or a mixture thereof. In one embodiment, one or more spray-supporting solvents are independently selected from the group consisting of water, a lower alcohol with up to 5 carbons, such as ethanol and isopropyl alcohol, a lower ketone with up to 5 carbons, such as acetone, methylethylketone and 3-pentanone, or a mixture thereof.

In one embodiment, the therapeutic formulation comprises hydrocortisone, clotrimazole, zinc oxide, petrolatum, water, cetearyl alcohol, ceteareth-20, Sodium phosphate, and one or more spray components. In one embodiment, a sprayable composition comprises hydrocortisone, clotrimazole, zinc oxide, petrolatum, water, cetearyl alcohol, ceteareth-20, Sodium phosphate, and one or more spray components, wherein the formulation is sprayable as a liquid, gel or foam.

In some embodiments, a set of sprayable compositions are provided with 1) a treatment-area contacting first part comprising hydrocortisone, clotrimazole, petrolatum, water, cetearyl alcohol, ceteareth-20, Sodium phosphate and one or more spray components; and 2) a protective-layer second part comprising zinc oxide, petrolatum and one or more spray components, wherein the formulations are independently sprayable as liquids, gels or foams. The treatment-area contacting first part is sprayed directly on the treatment area, while the protective-layer second part is sprayed over the first part and the treatment area.

In one embodiment, one or more spray components of the treatment-area contacting first part are independently selected from the group consisting of gelling agents, foaming agents, and spray-supporting solvents. In one embodiment, one or more spray components of the protective-layer second part are independently selected from the group consisting of gelling agents, foaming agents, and spray-supporting solvents.

In some embodiments, the therapeutic formulations or sprayable compositions are delivered using manual pumps, propellant pumps, or as metered doses.

In other embodiments, Dab Applicators are provided with a higher fluid content for hard to reach, damaged areas, which prevent direct touching and risk of contamination, and avoid the use of latex.

In another embodiment, protective masks are provided in which the therapeutic formulation is embedded in the seal of a protective mask, such as those used by paramedics, in hospital, long term care homes, other occupations requiring protection. This helps treat skin break down caused by sweat and friction caused by prolonged use of occlusive mask (and other protective clothing), and provides protection and risk mitigation in infectious environments.

In some embodiments, a kit is provided comprising one or more separate creams. These separate creams can be applied separately, or blended together and then applied. In one embodiment, one of the creams is the therapeutic formulation described herein comprising hydrocortisone, clotrimazole, and zinc oxide, and optionally one or more of enzyme inhibitors, pH buffers, moisturizing agents or humectants, antimicrobial agents, antifungal agents, or peptides described herein. In some embodiments, two or more of the creams are blended together to prepare the therapeutic formulation described herein comprising hydrocortisone, clotrimazole, and zinc oxide. In one embodiment, the kit comprises three or four separate creams. In one embodiment, a first cream comprises non-medicated ingredients for skin protection. In one embodiment, a second cream comprises hydrocortisone ranging from 0.1% to 1.1% by weight, preferably 1%. In another embodiment, a second cream comprises hydrocortisone and zinc oxide. In one embodiment, a third cream comprises clotrimazole ranging from 0.1% to 1.1% by weight, preferably 1%. In one embodiment, a third cream comprises clotrimazole and zinc oxide. In some embodiments, the second and third creams are separate creams. In other embodiments, the second and third creams are manufactured together as a single product. In one embodiment, a fourth cream comprises one or more of enzyme inhibitors, pH buffers, moisturizing agents or humectants, antimicrobial agents, antifungal agents, or peptides described herein. In one embodiment, the first, second, and/or the third creams comprise one or more of enzyme inhibitors, pH buffers, moisturizing agents or humectants, antimicrobial agents, antifungal agents, or peptides described herein. In one embodiment, the fourth cream is formulated to specifically treat the issues with the risk factors associated with development of MASD in patients with diarrhea, and comprises one or more of: Soybean Tyrosine Inhibitor (TSI) at concentration of 0-25% by weight (i.e. 1% by weight); Birk-Bowman Inhibitor, Birk-Bowman Related Protease Inhibitors, and/or Birk-Bowman Family Protease Inhibitors at concentration of 0-25% (i.e. 1% by weight); or non-denatured soybean extract (i.e. 10% by weight).

In some embodiments, the compositions and formulations described herein are provided in freeze dried powder form, comprising 1-100% by weight said composition or formulations and the rest comprising Cornstarch, Tapioca starch, Arrowroot starch, Kaolin clay, Oat flour, Talcum, or combinations thereof. This powder is dry sprayed onto the cream formulation described herein that has been topically applied to the skin. This dry spray adheres to the topical and dries to create a crust or film that physically protects against additional incontinence episodes between cleanings. Dry or wet spray would include Anti-fecal peptides or any from the family of botanical protease inhibitors. Formulation would include an adherent, which could include any of the above dry powder agents.

### Treatment and Protocols

The present disclosure relates to compositions and methods for treating a subject to reduce MASD, e.g., surgical adhesions, and includes administering to a subject a therapeutic formulation as described herein. In some embodiments the MASD is intertrigo. In some embodiments the MASD is incontinence associated.

The compositions and formulations described herein may also be used in the prevention or treatment of tinea pedis, tinea cruris, and tinea corporis due to Trichophyton rubrum, Trichophyton mentagrophytes, Epidermophyton floccosum; (2) Candidiasis due to Candida albicans; (3) Tinea versicolor due to Malassezia furfur, or anorectal pruritus.

Methods include administering to a subject in need of treatment a therapeutically effective amount or an effective dose of the therapeutic formulation. In some embodiments, effective doses of the therapeutic entity described herein vary depending upon many different factors, including means of administration, target site, physiological state of the patient, whether the patient is human or an animal, other medications administered, and whether treatment is prophylactic or therapeutic. Usually, the patient is a human but nonhuman mammals can also be treated. Treatment dosages may be titrated to optimize safety and efficacy. Reduction in skin damage may be monitored.

The effective dose of an agent can vary with the agent, but will generally range from up to about 0.1 ml., 0.5 ml., 1 ml, 5 ml, 10 ml, 20 ml., 30 ml, 50 ml, or more depending on the surface area to be treated.

The therapeutic agent may be administered one or a plurality of days, and in some embodiments is administered daily, every two days, semi-weekly, weekly, etc. for a period of from about 1 to 2 weeks.

In some embodiments, for prophylactic applications, the therapeutic formulations are administered to a patient susceptible to, or otherwise at risk of MASD in an amount sufficient to eliminate or reduce the risk, lessen the severity, or delay the outset of the condition.

In other embodiments, for therapeutic applications, the therapeutic formulations are administered to a patient already suffering from MASD in an amount sufficient to cure, or at least partially arrest, the skin damage, including its complications and pathological sequelae. An amount adequate to accomplish therapeutic or prophylactic treatment is defined as a therapeutically- or prophylactically-effective dose.

In some embodiments, where the therapeutic formulations comprise enzyme inhibitors and/or pH buffers, the therapeutic formulations are administered to a patient suffering from urinary and/or fecal incontinence.

The pharmaceutical compositions are generally formulated in full compliance with all Good Manufacturing Practice (GMP) regulations of the U.S. Food and Drug Administration.

In some embodiments, the therapeutic formulations are administered to a patient in combination with biofilm treatment, such as employing high osmolarity conditions and/or surfactants. Exemplary biofilm treatments are described in WO2013052958, U.S. Ser. No. 10/021,876, U.S. Pat. Nos. 7,976,875, and 7,959,943, the entire contents of which are incorporated herein as reference.

In some embodiments, cornstarch is applied following application of the compositions or therapeutic formulations described herein to prevent removal of said compositions or therapeutic formulations. In one embodiment, cornstarch is applied by blowing or dry spraying (i.e. using a squeeze bottle). After applying the composition or therapeutic formulation onto an area of skin, cornstarch is blown or dry sprayed onto the area and adheres to the composition or therapeutic formulation, forming a natural protective film while also preventing removal of the composition or therapeutic. The natural protective film protects the skin against diarrhea. While covered with the cornstarch film, the area of skin with said applications can be cleaned with wipes and, when required, the cornstarch film can be removed with mineral oil. The skin below can then be cleaned, observed and the composition or therapeutic formulation can be reapplied, as needed. The cornstarch film can be reapplied, as needed.

A wide variety of skin care products are available to treat MASD; however, this wide variety of skin care products renders it difficult for the bedside nurse or caregiver to select the optimal product for a given situation. This challenge is exacerbated by a lack of consistency in product names and absence of a grading scale designed to aid clinicians when selecting products based on severity of IAD, irritant source, and presence of complications. (Doughty 2012). Anecdotally, often due to lack of assessment, poor understanding of barrier products and care planning, patients have a combination of products applied to the skin which may result in deterioration in its condition (Mahoney et al in 2019). Due to the lack of well-established clinical trials, recommendations about MASD are based on expert opinion, consensus statements and best practice. Gentle cleansing, use of hydrating topical agents and application of barrier products are the main elements in the prevention and treatment of IAD (Beele H 2018). IAD was the subject of Cochrane Systemic Review 2016 to clarify the effects of various skin care products and procedures for the treatment and prevention of IAD. They found no evidence that one skin care product was better than another.

In accordance with the present disclosure, exemplary protocols are provided for improved treatment of MASD using the compositions and therapeutic formulations provided herein.

### Example C-Protocol 1

Four individual medicated and non-medicated topical cream products used either individually or in combination. Cream A is non-medicated, used daily to protect skin and prevent MASD. Cream B is medicated (comprising hydrocortisone ranging from 0.1% to 1.1% by weight, preferably 1% and optionally zinc oxide) and applied for 7 days if there are signs of persistent redness or itchiness, pain, or burning. Area of skin where cream B is applied is further covered by cream A, if added skin protection is needed. When rash has healed, resume daily use of cream A only. Cream C is medicated (comprising clotrimazole ranging from 0.1% to 1.1% by weight, preferably 1% and optionally zinc oxide) and applied for 7 days if there is intense itching, especially between skin folds, or flaky skin or small red dots at the edge of rash or fungal infection suspected. Area of skin where cream C is applied is further covered by cream A, if added skin protection is needed. When rash has healed, resume daily use of cream A only. Cream D is non-medicated (comprising one or more of enzyme inhibitors, pH buffers, moisturizing agents or humectants, antimicrobial agents, antifungal agents, or peptides) and used for 7 days if there is liquid diarrhea resulting in redness, itching, pain, burning. Area of skin where cream D is applied is further covered by cream A, if added skin protection is needed. When rash has healed, resume daily use of cream A only.

### Example D—Additional Combination Protocol 3

Three or four medicated and non-medicated topical cream products used either individually or in combination. Cream A is as described in Example C. Creams B & C as described in Example C may be manufactured together as a single product and applied for 7 days if there is persistent and/or intense redness, itchiness, pain or burning. Area of skin where cream B&C is applied is further covered by cream A, if added skin protection is needed. When rash has healed, resume daily use of cream A only. Cream D is as described in Example C, and may be manufactured individually or combined with B, with C, or with B & C combined and applied for 7 days if there is persistent and/or intense redness, itchiness, pain or burning. Area of skin where cream D is applied is further covered by cream A, if added skin protection is needed. When rash has healed, resume daily use of cream A only.

### Example E—Alternative Protocol 2

Equal amounts of Cream A, B and C, or A, B, C and D as described in Example C may be combined and applied for 7 days in a single-step application when it's necessary to speed healing, minimize caregiver time and prevent skin damage from application of multiple agents. When rash has healed, resume daily use of cream A only.

A rabbit model of IAD was also investigated. A combination cream (DGBC) was used to assess healing of an animal model of IAD compared with three active components of the cream. Twenty-six IAD lesions were induced and each lesion was treated with the individual components and the combination cream. While the combination cream involving three agents was synergistically the fastest to heal, all single agent treatments arms were effective in the treatment of IAD with complete healing of all lesions at day 5 of treatment.

### Example F—Incontinence-Associated Dermatitis Rabbit Model

Rabbits were purchased from Charles River to be used for these experiments. Rabbits were chosen because they have large, relatively hairless ears suitable for topical application of IAD-inducing agents as well as treatment for incontinence-associated dermatitis (IAD). Additionally, the animals' ears were used to maximize the sample size, effectively halving the number of animals required for the project. Twenty-two female rabbits were purchased: 2 for the pilot phase and 18 for phase 2 of the project. The study was conducted, and animals were housed at the AVC Research Facility as per CCAC guidelines. The experimental treatment utilized a therapeutic cream composed of 1.0% hydrocortisone, 1.0% clotrimazole and 15.0% zinc oxide (DGBC).

Phase 1 pilot: To determine the most appropriate compound to induce IAD, one ear was IAD generated with 5% pancreatin (a fecal enzyme) and the other ear with 1% ammonium hydroxide (synthetic urine). Sterile 3×3 gauze was used as a carrier for the irritant. The gauze was saturated with 5 ml of artificial urine or pancreatin. This volume of irritant was sufficient to saturate each gauze. The saturated gauze was covered with an occlusive dressing (Opsite) and left in place for a period of 72 hours. An Elizabethan collar was placed on any rabbit that attempts to dislodge or remove the covered gauze. After 72 h, the occlusive dressings and gauze were removed. Treatments were applied in order vertically on the ear, and the order was rotated for each rabbit: Rabbit 1 (R1): A (BDGC therapeutic cream), B (clotrimazole), C (hydrocortisone), D (zinc oxide); Rabbit 2 (R2): D, A, B, C. Results are summarized in Table 1.

TABLE 1
Rabbit 1 and 2 results from Phase 1 study

| | | Wound Assessment | | | | | | |
|---|---|---|---|---|---|---|---|---|
| | | Persistent | Skin Loss | | | | Pain | PP |
| Day | IAD | redness (%) | (%) | Oedema | Maceration | Infection | scale | Score |

Rabbit 1:

| | | | | | | | | |
|---|---|---|---|---|---|---|---|---|
| 1 | 2B | 100 | 10 | Present | Present | Present | 1/15 | 1/4 |
| 2 | 2A | 80 | 10 | | Present | | 0/15 | 0/4 |
| 3 | 2A | 40 | 0 | | | | 0/15 | 0/4 |
| 4 | 1A | 0 | 0 | | | | 0/15 | 0/4 |
| 5 | No | 0 | 0 | | | | 0/15 | 0/4 |

Rabbit 2:

| | | | | | | | | |
|---|---|---|---|---|---|---|---|---|
| 1 | 2A | 100 | 5 | Present | Present | Present | 0/15 | 1/4 |
| 2 | 1B | 80 | 5 | Present | Present | Present | 0/15 | 0/4 |
| 3 | 1A | 30 | 2 | | Present | | 0/15 | 0/4 |
| 4 | No | 0 | 0 | | | | 0/15 | 0/4 |
| 5 | No | 0 | 0 | | | | 0/15 | 0/4 |

IAD Categorization: 1A Persistent redness without clinical signs of infection, 1B Persistent redness with clinical signs of infection, 2A Skin loss without clinical signs of infection, 2B Skin loss with clinical signs of infection.

### Phase 2 Clinical Trial.

Induction of IAD. 1.Sedation of rabbit: Midazolam 0.5 mg/kg intramuscularly (IM) plus buprenorphine 0.03 mg/kg IM. Animals were assessed for sedation prior to gauze application. Animals requiring further sedation were sedated with ketamine 5 mg/kg IM. 2.Cleaning of the ear: The skin of both inner ears of each animal were cleaned with a 2×2 gauze saturated with 3 ml isopropyl alcohol and allowed to air dry for 5 minutes before applying the saturated gauze. The animal were held for a period of 5 min to ensure the ear was dry prior to application of IAD induction. 3.Application of gauze with 5% pancreatin to induce IAD: both ears of the animals were irritated as follows: Sterile 2×2 gauze was used as a carrier for the irritant. The gauze was saturated with 3 ml of pancreatin. 4.Wrapping of the ear: Both ears of each animal had gauze with pancreatin applied to the medial surface. The saturated gauze was covered with an occlusive dressing (OpSite), and a loosely applied VetWrap over roll gauze and left in place for a period of 24 hours. 5.Monitoring period of 24 h: After 24 hours the bandage and occlusive dressing was removed and the ears assessed for sufficient irritation. If insufficient irritation is evident a new 2×2 gauze was saturated with 3 ml pancreatin and reapplied as above. 6. Removal of wrapping and gauze: Once sufficient irritation was established, the occlusive dressings and gauze was removed. 7.Sufficient Irritation for Treatment: A first visual assessment of the skin damage was performed utilizing an assessment of erythema and edema, as well as the Ghent Global IAD Categorization Tool. The categorization of the lesion considered ‘redness’, ‘skin loss’ and percentage of lesion area as a percentage. A photograph of the area of skin damage was taken. Ghent scale document was used in the pilot study was utilized for attachment irritation assessment. 8. Pain Management: Rabbits were treated with gabapentin 20 mg/kg PO once daily during IAD induction and the first day of treatment. Rabbits assessed as painful 1 hour after administration of gabapentin were treated with buprenorphine 0.03 mg/kg SC and be reassessed for pain in 1 hour.

Treatment: 1.Photograph collection: Day “n” for both ears: rabbits are held by the same researcher in the same area of the same room while the second researcher takes a photograph of the irritation of each ear. 2. The photograph and data for each ear and each animal are entered in the Ghent Scale document. 3.One researcher holds each rabbit while the other researcher delineates and marks the IAD irritation area utilizing the same standard ruler (in attachment). The IAD area is thus divided into four equal areas for treatment application. 4. Compounds are applied into designated areas of IAD on each ear. The four treatments will be as follows: DGBC therapeutic cream (A); 1.0% clotrimazole (B); 1.0% hydrocortisone (C), 15.0% zinc oxide (D). The sequence of treatment applied for each subject was randomized.

Synergy analysis: The synergy among the three compounds will be analyzed utilizing the Multi-dimensional Synergy of Combination (MuSyC), which is based on a two-dimensional (2D) extension of Hill Equation derived from mass action kinetics.

Analyses: Results will be analyzed considering the results for synergistic efficacy (b), synergistic potency (a), and synergistic cooperativity (g) for each compound. These three parameters will determine the synergist behaviour of the compounds

Adverse events/Risk: Hydrocortisone and clotrimazole are some of the most used medications on the World Health Organization (WHO) list of essential medicines [10]. Hydrocortisone, when applied, may cause local hypersensitivity reactions, skin irritation/pain, and atrophy of the skin upon prolonged use. Clotrimazole may cause blisters, discomfort/pain, edema, erythema, irritation, peeling/exfoliation, pruritus, rash, stinging/burning may occur in humans. Zinc Oxide may cause mild irritation upon topical application in animals.

Results: Twenty white Australian rabbits were purchased for Dr. Gordon's Bum Cream Clinical Trial. Two rabbits were utilized in the pilot protocol #21-039 for refinement of the main Clinical Trial. Eighteen female rabbits, weight 1.4±0.079 kg, were being used on protocol #21-051 with the goal of evaluating the safety, efficacy, and synergy of the therapeutic cream (DGBC). Incontinence-associated dermatitis (IAD) was artificially induced in 18 rabbits for the period of 24 h utilizing 1 ml of 5% pancreatin, occluded with Opsite on each ear. After reaching sufficient irritation (see results of Protocol #21-039), rabbits were treated with DGBC (A), 1% Clotrimazole (B), 1% Hydrocortisone (C) and 15% Zinc Oxide (D). Per protocol, the left and right ear were divided into four vertical sections (one for each compound) and treated with four compounds for a period of seven days. The sequence of compounds was randomized, and each rabbit received a specific and unique sequence. The median daily amount of DGBC therapeutic cream (A), 1% Clotrimazole (B), 1% Hydrocortisone (C), and 15% Zinc Oxide (D) was 0.13 g/d (range 0.1-0.15 g/d) over an area of 37 cm² (average ear area). A thin layer of each compound was applied in the demarked area. During the first three days of the clinical trial, rabbits were given gabapentin for pain; only three rabbits had a pain score of 1 after the third day, no further analgesia was given.

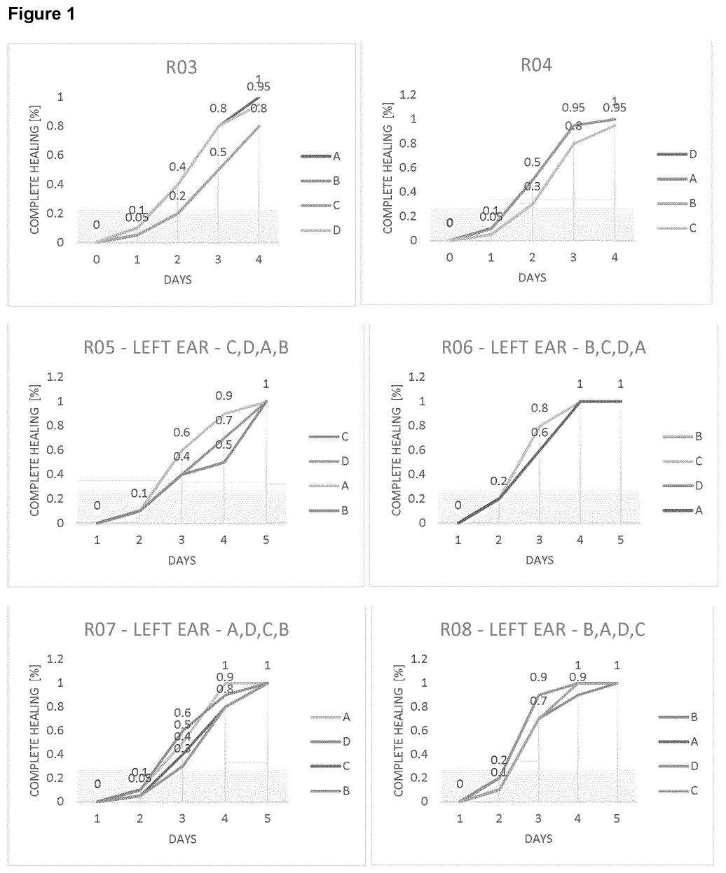

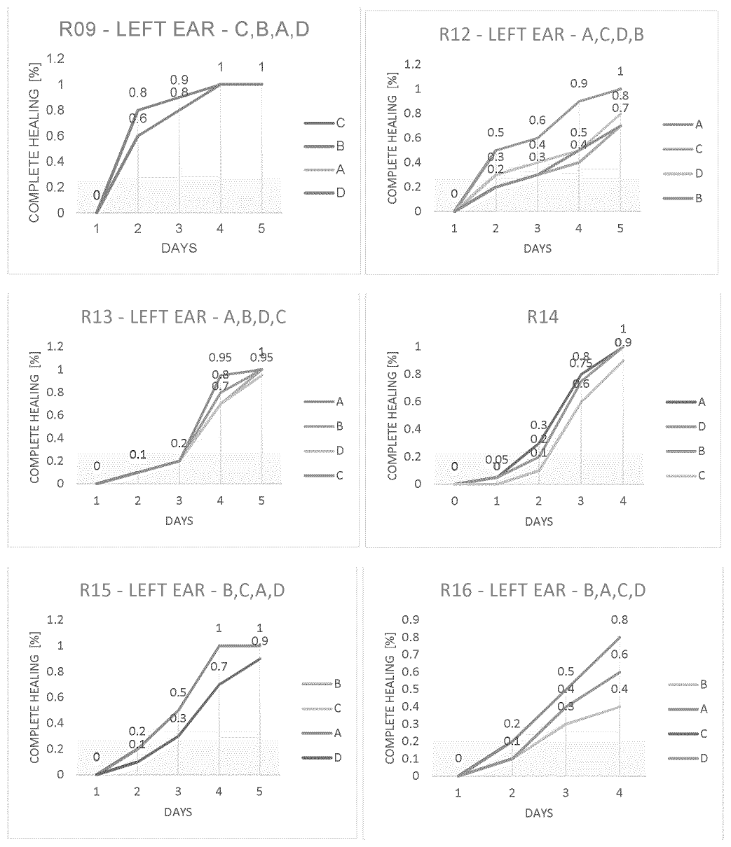

Efficacy. The results demonstrated that DGBC therapeutic cream provides complete healing in 70% of the rabbits, in average three days; 16.6% of the ears couldn't be assessed, and 13% of the ears were healed with a different compound than DGBC therapeutic cream, in some cases longer than seven days of treatment. The results demonstrated that hydrocortisone, clotrimazole, and zinc oxide separately do not produce the same effect as DGBC therapeutic cream with the combination of the three compounds. DGBC therapeutic cream showed superior results when utilized to treat IAD in rabbits, not having scabs or flaky skin after treatment compared to zinc oxide. See FIG. 1.

### Example G-Proteolytic Enzyme Assay Experiments

Methods. To develop a cream containing a raw soybean extract capable of inhibiting common digestive enzymes found in fecal matter, enzyme assay experiments were conducted. Pancreatin was used as the enzyme source, and Azocasein as the substrate to produce a compound which is UV active at 440 nm. The punative inhibitor of this reaction is a raw soybean extract. To measure the proteolytic activity of the pancreatin in solution, a spectrometric assay with Azocasein was used. The inhibitor was extracted in water from raw soybeans. Soybean extract was prepared by soaking an amount of soybean in purified water at 4° C. for 24 hours. The mixture was blended, centrifuged and supernatant retained. The soybean extract was incorporated into creams through a freeze-drying process. The creams tested were Dr Gordon's Bum Cream, Dr Gordon's Everyday Cream and a Petrolatum based cream. The creams were mixed with pancreatin and a protease inhibition assay was performed.

Results. The soybean extract, when added to a standard Pancreatin-Azocasein solution decreased absorption at 440 nm. The raw soybean extract inhibited the Pancreatin-Azocasein Reaction (PAR) by approximately 85% (Table 2). Tests were conducted to determine the inhibition by soybean extract with and without cream, using five different creams for the testing. For each cream, the cream was tested alone as a control comparator, and then the cream was tested with 3% soybean extract. Comparing the absorbance of the reaction at the working concentration and reaction with the cream, Azocasein and Pancreatin, it was observed that the reaction between the Azocasein and the Pancreatin took place without any interference from the cream (Table 3). The 3% soybean extract showed inhibition of the PAR in all creams.

TABLE 2
Responses and inhibition % of the assay reactions.

| | Absorbance (AU) | % inhibition |
|---|---|---|
| 1/100 dilution | 0.2535 | N/A |
| Pancreatin/Azocasein Rxn | | |
| (working concentration) | | |
| 0.5 mL Soybean extract | 0.0432 | 83.0 |
| 1.0 mL Soybean extract | 0.0348 | 86.3 |

TABLE 3
Responses of the extract of the cream and reaction
responses in contact with the cream.

| | Absorbance (AU) |
|---|---|
| 1/100 dilution Pancreatin/ | 0.1878 |
| Azocasein Rxn (working concentration) | |
| Reaction Assay with | 0.0098 |
| cream extract + Azocasein | |
| Reaction Assay with cream | 0.1834 |
| extract + Azocasein + Pancreatin | |

Table 2. To determine the percentage of inhibition of raw soybean extract, the Pancreatin-Azocasein (PAR) solution was prepared and incubated with two different volumes (0.5 mL and 1.0 mL) of the soybean extract. The raw soybean extract inhibited the PAR by approximately 85%.

Table 3. The interference of the cream alone at 440 nm and with Pancreatin-Azocasein (PAR) solution were investigated. The reaction was conducted with the Azocasein and 1.0 ml of the cream extract without Pancreatin to determine if the cream would react with Azocasein, and with PAR and the cream extract. Comparing the absorbance of the reaction at the working concentration and reaction with the cream, Azocasein and Pancreatin, it was observed that the reaction between the Azocasein and the Pancreatin took place without any interference from the cream.

Raw soybean extract was concentrated by evaporation to a thick paste by heating at 50° C. for about three days. Three cream samples with concentrated soybean extract were prepared: 3%, 7%, 15% by weight. The reaction Assays were repeated with Azocasein and Pancreatin with 1 g of cream at each concentration Table 4 shows the results for the % Inhibition for each cream.

TABLE 4
Responses of the reaction assays with
different % of Soybean in the cream.

| | Absorbance (AU) |
|---|---|
| Cream without Soybean | 0.1468 |
| extract | |
| Cream extract with 3% | 0.0525 |
| Soybean | |
| Cream extract with 7% | 0.0526 |
| Soybean | |
| Cream extract with 15% | 0.0592 |
| Soybean | |

Table 4. The degree of inhibition was similar at all three concentrations of soybean extract. The absorbance's were between 0.05-0.06 AU, with an overall inhibition of the Pancreatin-Azocasein Reaction (PAR) of 65-70%.

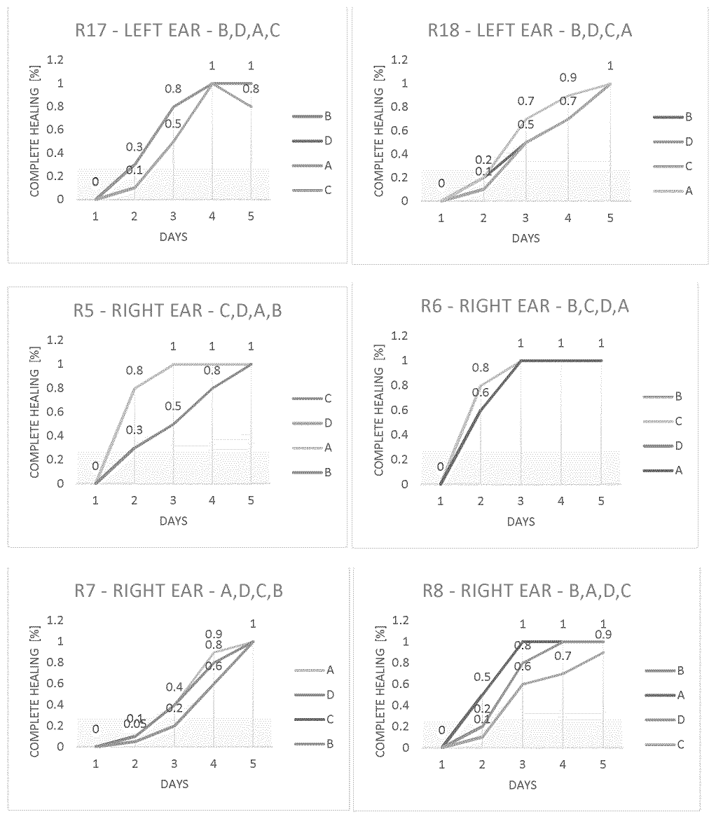

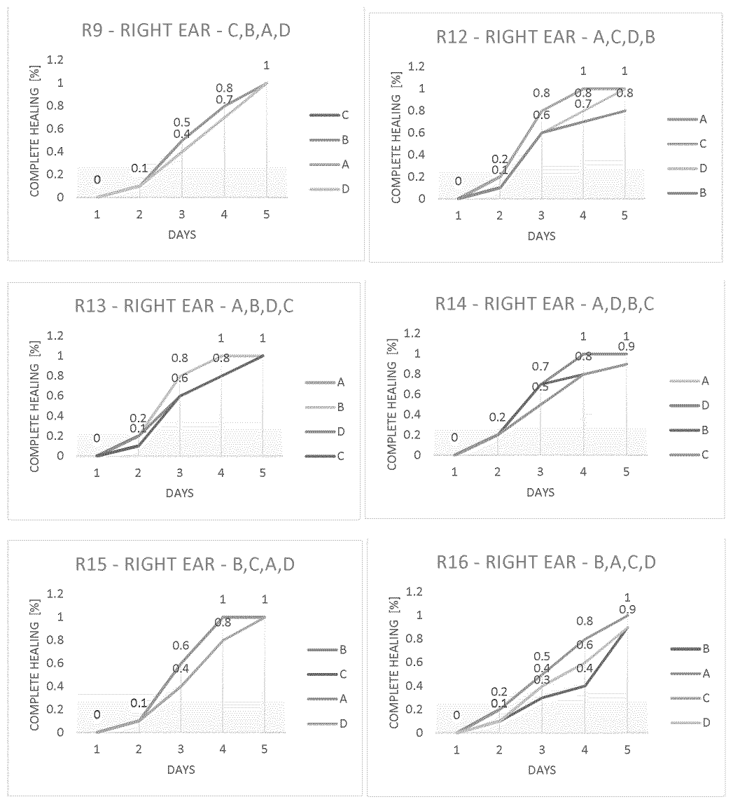

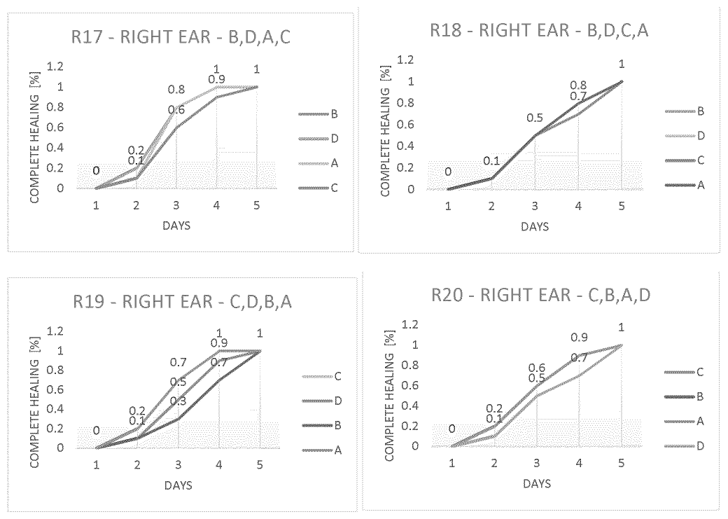

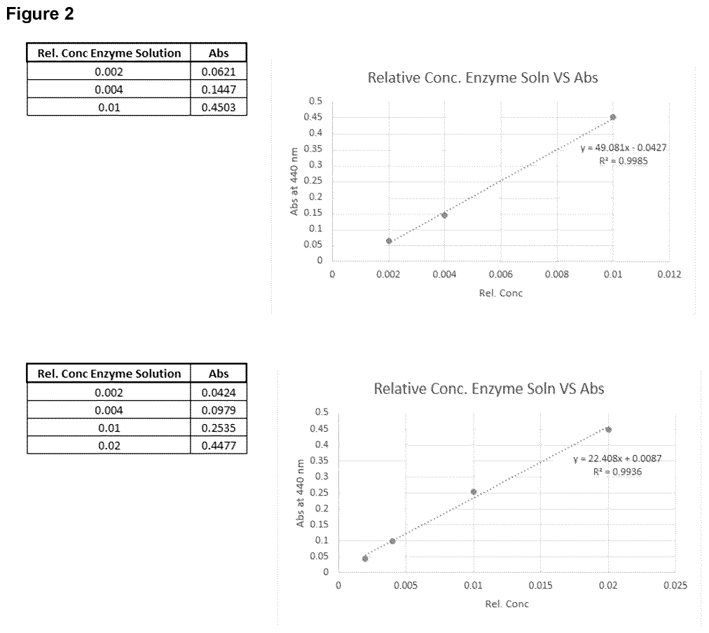

Soybean Inhibition. Two linear regression analysis were run looking at the relative concentration of enzyme solution vs absorption at 440 nm (see FIG. 2). The R-squared value is a statistical measure of how well a statistical model predicts an outcome. The R-squared value was 0.998 in the first regression analysis and 0.994 in the second regression analysis. As the relative concentration of the enzyme solution increased, the absorption at 440 nm increased in an almost linear manner.

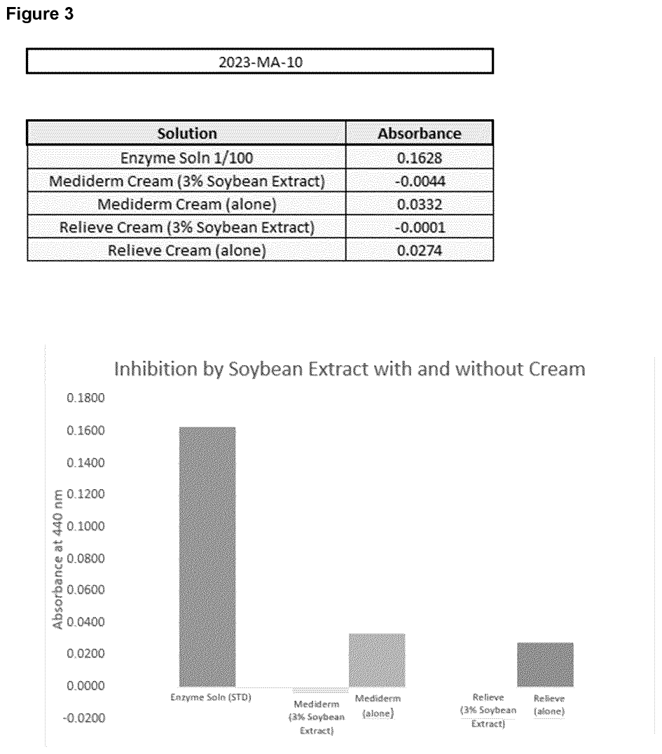

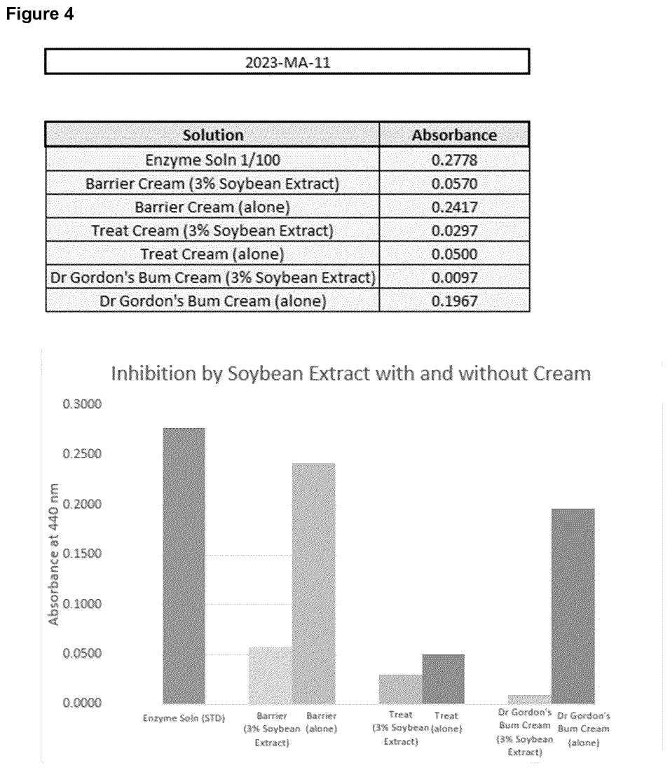

Tests were also conducted to determine the inhibition by soybean extract with and without cream, using five different creams for testing. A test of the PAR (Pancreatin-Azocasein Reaction) in solution alone was conducted as a control. For each cream, the cream was tested alone as a control comparator, and then the cream was tested with 3% soybean extract. The difference in absorbance at 440 nm was analyzed. The 3% soybean extract showed inhibition of the PAR in all creams: Mediderm 87%, Relieve 99%, Dr Gordon's Bum Cream 67%, Treat 77%, Dr Gordon's Everyday Cream 97%. See FIGS. 3 and 4.

### Example H—Natural Formulations

Cream D or other Natural Product in various embodiments including the dry spray would include: 1-40% by weight Zinc Oxide and 0.5-100% by weight Soy extract (or any other extracts from botanical family, such as those listed in Table A). As well as others agents selected from: Acetic acid, zinc salt-zinc acetate, Adeps Solidus-hard fat, Aluminum hydroxide, Avena Sativa
colloidal oatmeal, Carbamide, Carbonyldiamide, Carbonic acid sodium salt-baking soda, Carbonic acid, zinc salt-zinc carbonate, 2,5-Dioxo-4-imidazolidinyl-urea-allantoin, Iron oxide with zinc oxide-calamine, Kaolin (various), DL-Lactic acid, 2-Hydroxy-2 methylacetic acid-lactic acid, Lanolin, Anhydrous Lanolin-lanolin, wool fat, Olea europaea-olive oil, 1,2,3-Propanetriol
glycerine, Prunus dulcis-almond oil, Theobromo cacao-coca butter, Zea mays-cornstarch, Cod liver oil.

Treat Formulation: Aqua, Cetostearyl Alcohol, Clotrimazole, Glyceryl Laurate and Benzyl Alcohol, Paraffinum Liquidum, Polyethylene glycol monohexadecyl ether, and White Petrolatum. Treat Formulations may include additional components or comprise a combination of above listed components.

Relieve Formulation: 1% Hydrocortisone, Aqua, Cetostearyl Alcohol, Glyceryl Laurate and Benzyl Alcohol, Paraffinum Liquidum, Polyethylene glycol monohexadecyl ether, White Petrolatum, and aloe vera (i.e. Aloe Barbadensis leaf juice). Relieve Formulations may include additional components or comprise a combination of above listed components.

### Example I—Individual Case Study Data In Adult and Infant Populations

Preliminary results indicate that the cream is effective for relieving intense skin itching resulting from contact with feces and diarrhea. Preliminary evidence points to rapid relief of pain and itching associated with enterocutaneous fistula. Preliminary results indicate cream appears to reduce and relieve dermatitis associated with chronic diarrhea in infants. Cream was well tolerated. Comments included: ‘pleasant’, ‘not sticky’, ‘long-lasting’.

Case Study #1. In an individual with chronic symptoms resulting from anal surgery, they indicated that a single application daily was sufficient to relieve chronic anal itching.

Case Study #2. In an individual with a flaring of Crohn's Disease with persistent diarrhea for 72 hours, the cream was applied after each bowel movement. This flaring would normally result in significant perianal itching and burning. Individual reported the cream appears to have prevented skin damage as there was no redness or itching during or after this flare up.

Case Study #3. In an infant with chronic diarrhea and visibly red, irritated skin, following 3 days of application of the cream with each diaper change, the infant's perineal rash resolved. Parent noted there was rapid skin improvement and the child appeared calmer with diaper changes.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A therapeutic composition comprising:
from about 10% to about 15% by weight zinc oxide;
1% by weight hydrocortisone;
from about 0.5% to about 1% by weight clotrimazole;
optionally from about 40% to 50% by weight petrolatum;
optionally from about 0.01% to about 2.5% by weight dimethicone; and
optionally about 0.1-30% by weight a proteolytic enzyme inhibitor, preferably the proteolytic enzyme inhibitor is a trypsin inhibitor.

2.-17. (canceled)

18. The therapeutic composition of claim 1, wherein the composition comprises the proteolytic enzyme inhibitor, wherein the proteolytic enzyme inhibitor comprises Soybean Trypsin Inhibitor (STI), Birk-Bowman Inhibitor (BBI), non-denatured soybean extract, or a combination thereof.

19. (canceled)

20. The therapeutic composition of claim 1, comprising:
a) one or more non-active ingredients selected from the group consisting of cetearyl alcohol, mineral oil, lanolin, paraffin, water, sodium phosphate, EDTA, modified corn starch, and chlorocresol; and/or
b) one or more additives, and wherein the additives are pH buffers, humectants, antimicrobial agents, antifungal agents, or peptides.

21. The therapeutic composition of claim 1, comprising:
a)
12.5% by weight zinc oxide;
1% by weight hydrocortisone;
0.5% by weight clotrimazole;
0.05% by weight dimethicone; and
85.95% by weight petrolatum;
b)
15% by weight zinc oxide;
1% by weight hydrocortisone; and
1% by weight clotrimazole;
or c)
45% by weight petrolatum;
4% by weight cetearyl alcohol and ceteareth-20;
11% by weight mineral oil;
2% by weight lanolin;
4.5% by weight parafin;
10% by weight water;
0.2% by weight sodium phosphate, dibasic heptahydrate;
0.2% by weight disodium EDTA;
6% by weight modified corn starch;
15% by weight zinc oxide;
1% by weight clotrimazole;
1% by weight hydrocortisone; and
0.1% by weight chlorocresol.

22.-23. (canceled)

24. A kit comprising:
optionally a first composition preferably being a first cream comprising non-medicated ingredients for skin protection;
a second composition preferably being a second cream comprising 0.1-1.1% by weight hydrocortisone and optionally zinc oxide;
a third composition preferably being a third cream comprising 0.1-1.1% by weight clotrimazole and optionally zinc oxide; and
optionally a fourth composition preferably being a fourth cream comprising one or more of a pH buffer, a moisturizing agent or humectant, an antimicrobial agent, or an antifungal agent;
wherein optionally one or more of the first, second, third and fourth cream comprise a proteolytic enzyme inhibitor.

25.-26. (canceled)

27. The kit of claim 24 comprising about 0.1-30% by weight the proteolytic enzyme inhibitor, preferably wherein the proteolytic enzyme inhibitor is a trypsin inhibitor.

28. The kit of claim 24, wherein the fourth cream comprise the proteolytic enzyme inhibitor, wherein the proteolytic enzyme inhibitor comprises Soybean Trypsin Inhibitor (STI), Birk-Bowman Inhibitor (BBI), non-denatured soybean extract, or a combination thereof.

29.-30. (canceled)

31. The kit of claim 24, wherein the second and/or third cream comprises one or more of a pH buffer, a moisturizing agent or humectant, an antimicrobial agent, or an antifungal agent.

32. The kit of claim 24, wherein:
the second cream comprises 1% by hydrocortisone and zinc oxide; and/or
the third cream comprises 1% by clotrimazole and zinc oxide.

33. (canceled)

34. The kit of claim 24, wherein the second and third creams are blended together prior to application to skin.

35. (canceled)

36. The therapeutic composition of claim 1:
i. packaged in a sealed container;
ii. embedded in a disposable wipe;
iii. packaged in an aerosol, spray pump or continuous spray container;
iv. packaged in a dab applicator;
v. embedded in the seal of a protective mask; or
vi. applied as a biofilm.

37. A sprayable composition comprising the therapeutic composition of claim 1 and one or more spray components, wherein the one or more spray components comprise one or more of gelling agents, foaming agents, or spray-supporting solvents.

38. (canceled)

39. The kit of claim 24, wherein the second and third compositions are formulated together as a first sprayable composition and the kit further comprises a second sprayable composition, wherein:
the first sprayable composition comprises hydrocortisone, clotrimazole, petrolatum, water, cetearyl alcohol, ceteareth-20, Sodium phosphate and one or more spray components; and
the second sprayable composition comprises zinc oxide, petrolatum and one or more spray components;
wherein the one or more spray components of the first and second sprayable composition comprise gelling agents, foaming agents, or spray-supporting solvents.

40. (canceled)

41. A method for prevention or treatment of moisture-associated skin damage (MASD) in a subject, comprising:
topically applying to the skin of the subject from 1 to 100 g of a composition according to claim 1.

42. A method for prevention or treatment of moisture-associated skin damage (MASD) with the kit of claim 37 [or 38], comprising:
spraying the first sprayable composition over a treatment area of a subject; and
spraying the second sprayable composition over the first sprayable composition and the treatment area.

43. The method of claim 41, wherein the MASD is intertrigo or incontinence-associated dermatitis (IAD).

44. The method of claim 42, wherein the MASD is intertrigo or incontinence-associated dermatitis (IAD).

45. The method of claim 44, wherein the IAD is urinary incontinence, fecal incontinence, or combined urinary and fecal incontinence.

46.-50. (canceled)

51. A composition comprising a trypsin inhibitor and a carrier, for prevention or treatment of moisture-associated skin damage (MASD).

52. The composition of claim 51, further comprising clotrimazole, hydrocortisone, and/or zinc oxicde.

53. The composition of claim 51 being a sprayable composition, and optionally comprising zinc oxide.

54. The method of claim 43, wherein the IAD is urinary incontinence, fecal incontinence, or combined urinary and fecal incontinence.

55. A method of inhibiting fecal enzymes on a skin of a subject, the method comprising:
topically applying to the skin of the subject from 1 to 100 g of a composition according to claim 1.

56. A method of inhibiting fecal enzymes on a skin of a subject, the method comprising:
topically applying to the skin of the subject one or both of the second and the third composition defined in the kit of claim 24 sequentially or in combination; and
optionally applying the fourth composition defined in the kit of claim 24.

57. A composition comprising a trypsin inhibitor and a carrier, for inhibiting fecal enzymes on a skin of a subject.
